(19) United States
(12) Patent Application Publication
(10) Pub. No.: US 2011/0009852 A1
(43) Pub. Date: Jan. 13, 2011

(54) Laser Safety Goggles for Animals

(75) Inventors: Brian Pryor, NEWARK, DE (US); SEAN XIAOLU WANG, NEWARK, DE (US)

Correspondence Address: FRANK F. TIAN, 1300 SW Campus Drive No.5-7, Federal Way, WA, 98023, US

(73) Assignee: BWT PROPERTY, INC., Newark, DE (US)

(21) Appl. No.: 12/884,017

(22) Filed: Sep. 16, 2010

Related U.S. Application Data
(63) Continuation of application No. 12/037,146, filed on Feb. 26, 2008.
(60) Provisional application No. 60/891,734, filed on Feb. 27, 2007.

(51) Int. Cl. A61B 18/20; A61F 9/00

(52) U.S. Cl. 606/10; 2/15

(57) ABSTRACT

A laser protection device that can stay tightly on an animal by going over its head and eyes using a flexible material such as nylon. The device has polymer or glass windows that are designed to block or attenuate the treatment light in order to protect the eyes of the animal from laser damage. As an integral feature, the windows are partially transparent in the visible spectrum to allow the animal to see its surroundings.

### REFERENCE TO RELATED APPLICATIONS

This application is a continuation of (and claims the benefit of) an invention which was disclosed in non-provisional utility patent application Ser. No. 12/037,146, filed Feb. 26, 2008, entitled “Laser Safety Goggles for Animals,” which was in turn disclosed in Provisional Patent Application No. 60/891,734, filed Feb. 27, 2007, entitled “Laser Safety Goggles for Animals.” The benefit under 35 USC §119(e) of the above mentioned United States Provisional Applications is hereby claimed, and the aforementioned applications are hereby incorporated herein by reference.

### FIELD OF THE INVENTION

This invention generally relates to laser protection apparatus, and more specifically to laser safety goggles for animals.

### BACKGROUND

Animals are currently being treated with light based devices for medical treatments such as laser surgery and laser therapy. For these light based treatments, a safety apparatus is generally required to protect the eyes of the animals from direct or collateral light damage. Laser goggles made for human usage, such as those disclosed in U.S. Pat. Nos. 4,511,225, 5,708,490 and 6,081,934, will not fit animals since they will not stay on the animal nor do they have the appropriate shape to prevent the light from entering the animal eyes through their edges. Although there are devices such as eye blinders sold to block all visible light from entering the eyes of animals, these devices can not provide appropriate laser protection since many lasers used in medical treatments emit in the invisible spectrum and will penetrate these coverings. In addition, the eye blinder may cause the animal to be nervous during the treatment procedure.

### SUMMARY OF THE INVENTION

It is thus an object of the present invention to provide a laser safety goggle that will protect the animal eyes from laser radiation while still allowing the animal to see its surroundings. The goggle can easily stay on an animal by going over its head and eyes using a flexible material such as nylon. The goggle comprises polymer or glass windows that are designed to block or attenuate lasers of certain wavelengths to avoid eye damage.

### BRIEF DESCRIPTION OF THE FIGURES

The accompanying FIGURE, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 shows one exemplary embodiment of the laser safety goggle which is used on a horse.

Skilled artisans will appreciate that elements in the FIGURE are illustrated for simplicity and clarity and have not necessarily been drawn to scale. For example, the dimensions of some of the elements in the FIGURE may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to laser safety goggles for animals. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

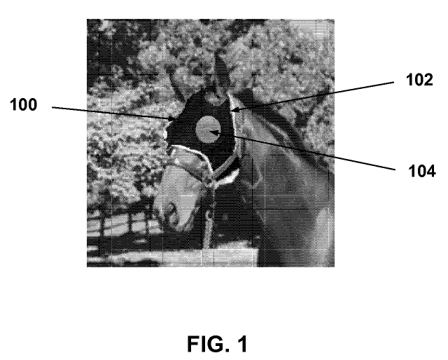

FIG. 1 shows one exemplary embodiment of the laser safety goggle which is used on a horse during laser treatment procedure. The laser safety goggle 100 comprises a flexible support member 102 made of nylon or any other suitable material, which can be easily tightened on the horse by going over its head and eyes. The laser safety goggle 100 further comprises two glass or polymer windows 104 built in the flexible support member 102 to cover the eyes of the horse. The glass or polymer windows 104 are doped or coated with appropriate material to absorb or reflect certain wavelength of light. The blocked or attenuated wavelength corresponds to the wavelength of the laser light to protect the eyes of the horse from potentially harmful light. As an integral feature, the glass or polymer windows 104 are transparent or partially transparent in the visible spectrum to allow the horse to see its surroundings. This is important when trying to keep the horse calm during the laser treatment.

In the foregoing specification, specific embodiments of the present invention have been described. However, one of ordinary skill in the art appreciates that various modifications and changes can be made without departing from the scope of the present invention as set forth in the claims below. Accordingly, the specification and FIGURE are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

What is claimed is:

1. A light treatment system for treating an animal, the light treatment system comprising:
a light source for producing treatment light to treat the animal; and
an eye protection apparatus for protecting the animal's eyes from treatment light irradiation, said eye protection apparatus comprising:
a flexible support member that can stay tightly on the animal by going over its head and eyes; and
windows built in said flexible support member to cover the eyes of the animal, wherein said windows are doped or coated with a material to block or attenuate a wavelength of light, and wherein said blocked or attenuated wavelength corresponds to a wavelength of said treatment light for eye protection.

2. The light treatment system of claim 1, wherein said light source comprises a laser.

3. The light treatment system of claim 1, wherein said windows are at least partially transparent in a visible spectrum to allow the animal to see its surroundings.

4. The light treatment system of claim 1, wherein said windows are made of glass or polymer materials.

5. A light treatment method for treating an animal, the light treatment method comprising the steps of:
providing a light source for producing treatment light to treat the animal; and
providing an eye protection apparatus for protecting the animal's eyes from treatment light irradiation, said eye protection apparatus comprising:
a flexible support member that can stay tightly on the animal by going over its head and eyes; and
windows built in said flexible support member to cover the eyes of the animal, wherein said windows are doped or coated with a material to block or attenuate a wavelength of light, and wherein said blocked or attenuated wavelength corresponds to a wavelength of said treatment light for eye protection.

6. The light treatment method of claim 5, wherein said light source comprises a laser.

7. The light treatment method of claim 5, wherein said windows are at least partially transparent in a visible spectrum to allow the animal to see its surroundings.

8. The light treatment method of claim 5, wherein said windows are made of glass or polymer materials.
